(19) United States
(12) Plant Patent Application Publication
(10) Pub. No.: US 2005/0262606 P1
(43) Pub. Date: Nov. 24, 2005

(54) Bougainvillea plant named 'Wabag'

(75) Inventors: Peter Iredell, Moggil Brisbane (AU); Jan Iredell, Moggil Brisbane (AU)

Correspondence Address: Catherine Anne Whealy;Proprietary Rights International, P.O. Box 220, Tolar, TX, 76476, US

(73) Assignee: Bougainvillea Nursery, Queensland (AU)

(21) Appl. No.: 10/973,063

(22) Filed: Oct. 23, 2004

(30) Foreign Application Priority Data
May 24, 2004 (AU) 2000/347

(51) Int. Cl. A01H 5/00

(52) U.S. Cl. PLT/256

(57) ABSTRACT

A new and distinct cultivar of Bougainvillea plant named ‘Wabag’, characterized by its outwardly spreading and bushy plant habit; vigorous growth habit; green and pale yellow variegated leaves; freely flowering habit; and red-colored flower bracts.

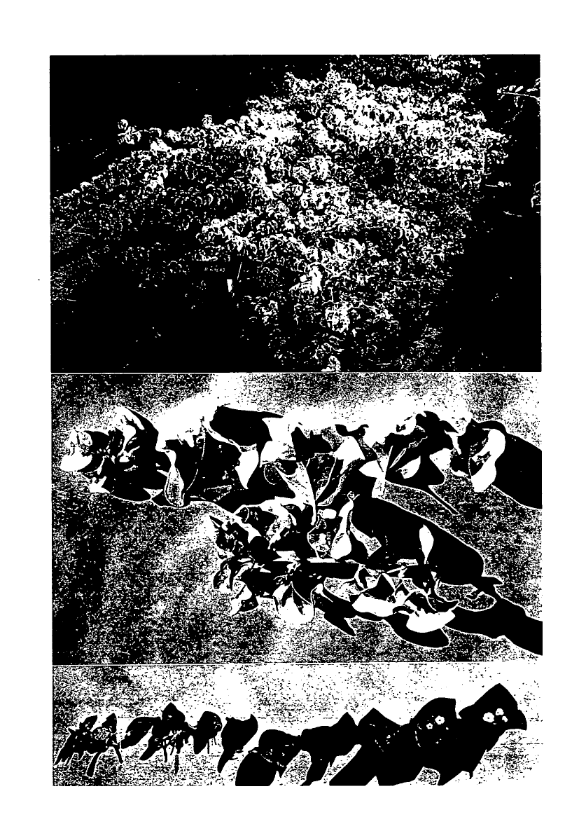

### BOTANICAL CLASSIFICATION/CULTIVAR DESIGNATION

Bougainvillea hybrida cultivar Wabag

### BACKGROUND OF THE INVENTION

The present Invention relates to a new and distinct cultivar of Bougainvillea plant, botanically known as Bougainvillea hybrida, and hereinafter referred to by the name ‘Wabag’.

The new Bougainvillea is a naturally-occurring branch mutation of the Bougainvillea hybrida cultivar Jazzi, not patented. The new Bougainvillea was discovered and selected by the Inventors from within a population of plants of the cultivar Jazzi in a controlled environment in Brisbane, Queensland, Australia in 1994. The selection of this plant was based on its variegated leaves.

Asexual reproduction ofthe new cultivar by cuttings at Brisbane, Queensland, Australia since 1995, has shown that the unique features of this new Bougainvillea are stable and reproduced true to type in successive generations.

### SUMMARY OF THE INVENTION

Plants of the cultivar Wabag have not been observed under all possible environmental conditions. The phenotype may vary somewhat with variations in environment such as temperature and/or light intensity without, however, any variance in genotype.

The following traits have been repeatedly observed and are determined to be the unique characteristics of ‘Wabag’. These characteristics in combination distinguish ‘Wabag’ as a new and distinct cultivar:

1. Outwardly spreading and bushy plant habit.

2. Vigorous growth habit.

3. Green and golden yellow variegated leaves.

4. Freely flowering habit.

5. Red-colored flower bracts.

Plants of the new Bougainvillea are most similar to plants of the parent, the cultivar Jazzi. In side-by-side comparisons conducted in Moggill, Queensland, Australia, plants of the new Bougainvillea differed primarily from plants of the cultivar Jazzi in leaf coloration as plants of the cultivar Jazzi did not have variegated leaves.

Plants of the new Bougainvillea can also be compared to plants of the Bougainvillea cultivar Bilas, not patented. In side-by-side comparisons conducted in Moggill, Queensland, Australia, plants ofthe new Bougainvillea differed primarily from plants of the cultivar Bilas in leaf coloration as plants of the cultivar Bilas had light green-colored leaves with golden speckles and spots.

### BRIEF DESCRIPTION OF THE PHOTOGRAPHS

The accompanying colored photographs illustrate the overall appearance of the new cultivar, showing the colors as true as it is reasonably possible to obtain in colored reproductions of this type. Colors in the photographs may differ from the color values cited in the detailed botanical description which accurately describe the colors ofthe new Bougainvillea.

The photograph at the top of the sheet is a side perspective view of typical one-year old plants of ‘Wabag’ grown in the landscape. The photograph in the middle of the sheet is a close-up view of typical developing flowers of ‘Wabag’. The photograph at the bottom of the sheet is a close-up view oftypical developing leaves and a typical lateral branch of ‘Wabag’.

### DETAILED BOTANICAL DESCRIPTION

In the following description, color references are made to The Royal Horticultural Society Colour Chart, 2001 Edition, except where general terms of ordinary dictionary significance are used. Plants used for the following botanical description were grown in Moggill, Queensland, Australia in a polyethylene-covered greenhouse. Plants used for the description were about one to two years old.

### Botanical Classification

Bougainvillea hybrida cultivar Wabag.

### Parentage

Naturally-occurring branch mutation ofthe Bougainvillea hybrida cultivar Jazzi, not patented.

### Propagation

Type: By cuttings.
Time to initiate roots: About one week at 25° C.
Time to develop roots: About two weeks at 25° C.
Root description: Thick; white in color.

### Plant Description

Form: Outwardly spreading and bushy plant habit; vigorous growth habit. Freely branching with lateral branches potentially developing at every node.
Plant height, soil level to top of plant plane: About 1 to 1.5 meters.
Plant width: About 1 to 1.5 meters.
Stem color: 161A.
Stem texture: Smooth, glabrous.
Foliage description:
   Arrangement.—Opposite, simple.
   Length.—About 8 cm.
   Width.—About 6.1 mm.
   Shape.—Broadly ovate.
   Apex.—Acuminate.
   Base.—Obtuse.
   Margin.—Entire; slightly undulate.
   Texture, upper and lower surfaces.—Smooth, glabrous.
   Venation pattern.—Pinnate.
   Color.—Developing foliage, upper surface: Towards the center, 137A; towards the margins, 160A. Developing foliage, lower surface: 137B. Fully expanded foliage, upper surface: Towards the center, 137A; towards the margins, 160A. Fully expanded foliage, lower surface: Towards the center, 143A; towards the margins, 161C. Venation, upper surface: 137B. Venation, lower surface: 160A.
   Petiole length.—About 2.1 cm.
   Petiole diameter.—About 1 mm.
   Petiole texture, upper and lower surfaces.—Smooth, glabrous.
   Petiole color, upper and lower surfaces.—160A.

### Flower Description

Flower type and habit: Single flowers arranged in axillary panicles with showy bracts. Flowers face mostly upright. Flowers not persistent.
Fragrance: None.
Natural flowering season: Cyclically flowering year-round in Moggill, Queensland, Australia; flowering periods about six to twelve weeks long.
Quantity: Freely flowering with about 30 flowers per panicle.
Flower longevity: About six to ten days.
Flower diameter: About 4 mm.
Flower depth (height): About 1.8 cm.
Flower buds:
   Length.—About 1.7 cm.
   Diameter.—About 2 mm.
   Shape.—Elongated.
   Color.—46B.
Petals:
   Quantity per flower.—About five; fused tubular corolla.
   Length.—About 1.7 cm.
   Width.—About 3 mm.
   Shape.—Fan-shaped.
   Apex.—Emarginate.
   Margin.—Entire.
   Texture, upper and lower surfaces.—Smooth; glabrous.
   Color.—When opening, upper and lower surfaces: 4D. Fully opened, upper and lower surfaces: 4D; color becoming closer to 159D with development.
Sepals:
   Quantity per flower.—About five; fused tubular corona.
   Length.—About 1.3 cm.
   Diameter.—About 2.5 mm.
   Shape.—Linear.
   Apex.—Acute.
   Margin.—Entire.
   Color.—When opening, upper and lower surfaces: 180B. Fully opened, upper and lower surfaces: 180B.
Flower bracts:
   Quantity per flower.—Three.
   Length.—About 3.6 cm.
   Width.—About 3 cm.
   Shape.—Broadly ovate.
   Apex.—Acute.
   Base.—Obtuse.
   Margin.—Entire; slightly undulate.
   Texture, upper and lower surfaces.—Smooth; glabrous; rugose.
   Color.—When opening, upper surface: 46B. When opening, lower surface: 45B. Fully opened, upper surface: 52B. Fully opened, lower surface: 45B.
Peduncles:
   Length.—About 2.5 cm.
   Diameter.—About 1 mm.
   Texture.—Smooth, glabrous.
   Angle.—About 45° from vertical.
   Strength.—Strong.
   Color.—180D.
Pedicels:
   Length.—About 1.2 cm.
   Diameter.—About 0.75 mm.
   Texture.—Smooth, glabrous.
   Angle.—About 45° from vertical.
   Strength.—Strong.
   Color.—180B.
Reproductive organs:
   Androecium.—Stamens per flower: About seven. Anther shape: Oblong. Anther length: About 0.5 mm. Anther color: 13A. Pollen color: 13A.
   Gynoecium.—Quantity of pistils per flower: One. Pistil length: About 1 cm. Stigma shape: Linear. Stigma color: 145C. Style length: About 5 mm. Style color: 145B. Ovary color: 145A.
Seeds/fruits: Seed and fruit development has not been observed.

### Disease/Pest Resistance

Under commercial production conditions, plants of the new Bougainvillea have not been noted to be resistant to pathogens or pests common to Bougainvillea.

### Garden Performance

Plants of the new Bougainvillea have good garden performance as plants of the new Bougainvillea have been observed to tolerate wind, rain and temperatures from 3 to 40° C.

1. A new and distinct cultivar of Bougainvillea plant named ‘Wabag’, as illustrated and described.
